(19) United States
(12) Patent Application Publication
(10) Pub. No.: US 2012/0064527 A1
(43) Pub. Date: Mar. 15, 2012

(54) NUCLEIC ACID ANALYSIS DEVICE, NUCLEIC ACID ANALYSIS APPARATUS, AND NUCLEIC ACID ANALYSIS METHOD

(76) Inventors: Akira Maekawa, Hitachinaka (JP); Tomoyuki Sakai, Kokubunji (JP); Tsuyoshi Sonehara, Kokubunji (JP); Satoshi Takahashi, Hitachinaka (JP)

(21) Appl. No.: 13/322,203

(22) PCT Filed: May 24, 2010
(86) PCT No.: PCT/JP2010/058710

§ 371 Date: Nov. 23, 2011

(30) Foreign Application Priority Data
May 27, 2009 (JP) 2009-127907

(51) Int. Cl. C12Q 1/68; G01N 33/50; C12M 1/34; G01N 21/64

(52) U.S. Cl. 435/6.11; 422/82.08; 436/94; 435/287.2; 435/6.1

(57) ABSTRACT

The present invention relates to a nucleic acid analysis device in a nucleic acid analysis apparatus, whereby waste of reaction spots on the nucleic acid analysis device is eliminated and leakage of fluorescence excitation light to unobserved nucleic acid measurement regions is minimized. Specifically, the nucleic acid analysis device has a plurality of nucleic acid measurement regions, which are characterized in that one nucleic acid measurement region is disposed at a sufficient distance from the other nucleic acid measurement regions such that the other nucleic acid measurement regions do not enter an irradiation region.

### TECHNICAL FIELD

The present invention relates to a nucleic acid analysis device and a nucleic acid analysis apparatus, for example.

### BACKGROUND ART

A sequencing method in a nucleic acid analysis apparatus has been recently proposed, which comprises immobilizing many DNA probes or polymerases on a reaction device prepared using a glass substrate or the like and then performing a base extension reaction on the reaction device. A region in which such immobilization and reaction are performed is hereinafter referred to as “reaction spot.”

A single molecule is immobilized (single molecule scheme) or a plurality of molecules of the same type are immobilized (multimolecular scheme) at a reaction spot. Also, a massively parallel nucleic acid analysis apparatus has also been developed, whereby many reaction spots are disposed and base extension and sequencing are performed in parallel in many reaction spots.

Non-patent document 1 explains a case in which a single molecule is immobilized at a reaction spot. Non-patent document 1 describes DNA sequencing using a total reflection evanescent irradiation detection system at the single molecule level. Specifically, a laser having an excitation wavelength of 532 nm and a laser having an excitation wavelength of 635 nm are used as excitation light for exciting fluorescence from fluorophore Cy3 and fluorophore Cy5, respectively. First, a single target DNA molecule is immobilized on the solution layer side on a refractive index boundary plane using biotin-avidin protein binding, forming a reaction spot. Cy3-labeled primers are introduced into a solution by solution exchange, so that the single fluorescence-labeled primer molecule hybridizes to the target DNA molecule. The hybridization reaction is performed for a certain time period, and then excessive primers that have remained unreacted are washed off. Subsequently, as a result of total reflection evanescent irradiation using excitation light (532 nm), since Cy3 is present in the evanescent field, the position of binding of the target DNA molecule can be confirmed by fluorescence detection. After the confirmation, Cy3 is irradiated with high-power excitation light for fluorescence photobleaching, thereby suppressing the subsequent fluorescent emission.

Next, polymerase and a single type of base labeled with Cy5, dNTP (N denotes A, C, G, or T), are introduced into a solution by solution exchange, the fluorescence-labeled dNTP molecule is incorporated into the extension chain of the primer molecule only when it is complementary to the target DNA molecule. The extension reaction is performed for a certain time period, and then excessive dNTP that has remained unreacted is washed off. Subsequently, as a result of total reflection evanescent irradiation using excitation light (635 nm), since Cy5 is present in the evanescent field, the complementary relationship can be confirmed by fluorescence detection at the binding position of the target DNA molecule. After confirmation, Cy5 is irradiated with high-power excitation light for fluorescence photobleaching, so as to suppress the subsequent fluorescent emission. In the above reaction process for incorporation of dNTP, a base sequence that is complementary to the target DNA molecule can be determined through stepwise extension reaction; that is, the repetition of the sequential use of base types, such as A→C→G→T→A→ . . . .

A plurality of reaction spots are formed within regions (hereinafter referred to as “visual measurement field(s)”) that can be observed simultaneously by a detector to be used for fluorescent measurement, and then the above reaction processes for dNTP incorporation are performed in parallel while different target DNA molecules are present in reaction spots. This enables simultaneous DNA sequencing for a plurality of target DNA molecules. It is expected that the number of subjects that can be simultaneously treated in parallel can be drastically increased compared with conventional DNA sequencing based on electrophoresis.

Also, a single molecule DNA sequencer does not require gene amplification by a PCR or the like, because of its mechanism. However, when a target DNA fragment to be observed is rare or only a single DNA fragment, a single molecule DNA sequencer can ideally read the target without wasting the DNA fragment.

Furthermore, there is a method in advanced research that uses a combination of semiconductor chips having microstructures for generation of plasmon resonance or the like in order to perform DNA sequencing (determination of the base sequence) for a single molecular unit. For example, Patent document 1 describes the use of the effects of enhancing fluorescence to a degree about several to dozens of times that of localized surface plasmons. The effect of enhancing fluorescence can reach the range of about 10 nm to 20 nm. When localized surface plasmons are generated on the surface of a metal microstructure to which a target DNA molecule has been immobilized, only the fluorescence-labeled dNTP incorporated into the target DNA molecule receives the benefit from the enhanced fluorescence, resulting in a difference in fluorescence intensity several to dozens of times or more greater than that for floating fluorescence-labeled dNTP. Such a scheme makes it possible to measure a base extension reaction without removing unreacted fluorescence-labeled dNTP.

Also, various methods have been proposed whereby target molecules are aligned in arbitrary shapes or at arbitrary positions. Non-patent document 2 proposes a method that involves firstly providing an electrode in a desired pattern on a substrate, coating the entire substrate surface with PLL-g-PEG (Poly-L-Lysin-g-polyethylene glycol), and applying voltage to the electrode, so as to remove PLL-g-PEG on the electrode part, and thus causing fluorescent molecules or the like to specifically adsorb to the electrode part alone. Non-patent document 3 describes a technique that involves coating a substrate with photodissociative molecules and then preparing an immobilization region pattern for nanoscale target molecules by a lithography technique using near-field scanning light. According to these techniques, a pattern of 100-nm or less DNAs or proteins is prepared on a substrate.

Meanwhile, Non-patent document 4 discloses real-time DNA sequencing analysis that involves supplying different fluorescent dyes to 4 types of nucleotide and causing serial nucleic acid extension reactions without washing. Also, Patent document 2 discloses a method for controlling the topical initiation of a base extension reaction, which involves disposing a protecting group cleavable by light irradiation at position 3′ of a probe. Specifically, a caged compound is disposed as a protecting group at position 3′ on the oligo probe, the protecting group is cleaved by UV irradiation, and then a real-time base extension reaction is initiated.

### PRIOR ART DOCUMENTS

Patent Documents
Patent document 1: JP Patent Publication (Kokai) No. 2009-45057 A
Patent document 2: JP Patent Publication (Kokai) No. 2010-48 A

Non-Patent Documents
Non-patent document 1: Ido Braslaysky et al., “Proc. Natl. Acad. Sci. U.S.A.,” 2003, Vol. 100, No. 7, pp. 3960-3964
Non-patent document 2: C. S. Tang et al., “Analytical Chemistry,” 2006, Vol. 78, No. 3, pp. 711-717
Non-patent document 3: Yasuhiro Kobayashi et al., “Analytical Sciences,” 2008, Vol. 24, No. 5, pp. 571-576
Non-patent document 4: John Eid et al., “Science,” Jan. 2, 2009, Vol. 323, No. 5910, pp. 133-138

### SUMMARY OF THE INVENTION

### Problems to be Solved by the Invention

The present inventors have obtained the following findings as a result of intensive studies to improve the throughput of a massively parallel nucleic acid analysis apparatus.

In the case of the massively parallel nucleic acid analysis apparatus, the throughput is improved proportionally to regions (specifically, the number of effective reaction spots within measurement regions) that can be measured simultaneously with a single optical detection system composed of a lens and a detector. Also, reaction spots are disposed in high density on a reaction device, so that the amount of a reagent to be used herein is reduced since the reaction chamber size becomes smaller even with the same number of reaction spots, and analysis with lower cost becomes possible. However, in reality, the number of simultaneously measurable effective reaction spots and the density of reaction spots in a reaction device are limited by the resolution of the optical detection system and the number of pixels of the detector.

The resolution of an optical detection system is determined on the basis of the diffraction limit of an objective lens composing the optical detection system. The diffraction limit is specifically determined according to the following equation.

Diffraction limit=0.61×λ/NA [Equation 1]

(where “λ” denotes the wavelength of light to be measured and “NA” denotes the numerical aperture of an objective lens.)

The wavelength of fluorescence to be measured ranges from about 500 nm to 800 nm, while the NA of an objective lens is about 1. According to the above equation, the diffraction limit of an objective lens ranges from about 300 nm to 500 nm. The resolution of an actual optical detection system is even lower than the above values because of the aberration, positional precision, and the like for a lens, such as about 1 μm. Accordingly, reaction spots must be at a distance of about 1 μm or more away from each other in order to ensure the identification of fluorescence on individual reaction spots. On the other hand, the range of the measurable visual field (effective visual field size) depends on the NA of an objective lens to be used. When the NA is about 1, the effective visual field size is about 1 mm². Hence, reaction spots should be formed with a pitch of 1 μm within a 1-mm² range in order to maximize the number of reaction spots within a measurement region. At this time, the maximum number of reaction spots is about 1×10⁶.

An even larger number of reaction spots should be formed to further improve the throughput. Hence, there is a method that involves forming 1×10⁶ or more reaction spots on a substrate and measuring while scanning.

When the above measurement is performed, the signal intensity should be suppressed within the dynamic range of a detector. Accordingly, excitation light intensity within the measurement region should be as uniform as possible. Thus, the excitation light irradiation region should be larger than the measurement region. Excitation light also leaks to reaction spots adjacent to a measurement region to be subjected to fluorescence measurement, so that light leakage occurs. In this case, a fluorescent dye is decomposed via irradiation with excitation light and quenching takes place, and quenching of fluorescence may be caused within the reaction spots adjacent to the measurement region to be subjected to fluorescence measurement. When the adjacent reaction spots are within unmeasured regions, in the case of a multimolecular scheme, some of fluorophores labeled with a plurality of molecules of the same type within the reaction spots or fluorophores labeled with molecules to be incorporated into a plurality of molecules of the same type are quenched by light leakage and thus sufficient signal intensity may not be obtained. This increases the noise information against the base sequence to be determined. In the case of the single molecule scheme, only one target molecule is present within a reaction spot, and the problem of quenching due to light leakage is more serious than in the multimolecular scheme.

A possible means for addressing the problem of fluorescence quenching due to light leakage is to conduct measurement for regions sufficiently distant from each other so that individual irradiation regions are not allowed to overlap each other. However, when a structure wherein measurement regions are sufficiently distant from each other is employed, target molecules within reaction spots existing between the measurement regions are not measured, and the information or target molecules existing in the region cannot be obtained.

In view of the above circumstances, an object of the present invention is to reduce reaction spot waste on a nucleic acid analysis device in a nucleic acid analysis apparatus, and to provide a nucleic acid analysis device by which the leakage of fluorescence excitation light to unobserved measurement regions is suppressed.

### Means for Solving the Problems

As a result of intensive studies to achieve the above object, the present inventors have found that the leakage of fluorescence excitation light to regions other than the target nucleic acid measurement region can be suppressed by disposing one nucleic acid measurement region so that it is at a sufficient distance from other nucleic acid measurement regions on a nucleic acid analysis device, and so that other nucleic acid measurement regions do not enter the irradiation region. Thus, the present inventors have completed the present invention.

Specifically, the present invention relates to a nucleic acid analysis device having a plurality of nucleic acid measurement regions wherein one nucleic acid measurement region is disposed so that it is at a sufficient distance from other nucleic acid measurement regions, and so that other nucleic acid measurement regions do not enter the irradiation region. Also, the present invention relates to a nucleic acid analysis apparatus comprising the nucleic acid analysis device and a nucleic acid analysis method using the nucleic acid analysis device.

This description includes part or all of the content as disclosed in the description and/or drawings of Japanese Patent Application No. 2009-127907, which is a priority document of the present application.

### Effects of the Invention

The present invention exerts an effect of reliably capturing fluorescence signals from target nucleic acids immobilized within the target nucleic acid measurement regions.

### BRIEF DESCRIPTION OF DRAWINGS

FIG. 1 is a schematic view illustrating an example of a nucleic acid analysis device and an optical detection system.

FIG. 2 is a schematic view of an example of an apparatus provided with a nucleic acid analysis device for determination of base sequences.

FIG. 3 is a schematic view showing an visual observation field in a nucleic acid analysis device after change.

FIG. 4 is a schematic view illustrating an example of the structure of a nucleic acid analysis device, having reagent flow channels therein.

FIG. 5 shows examples of parallel treatment steps performed in a plurality of reagent flow channels.

FIG. 6 is a schematic view showing an example of a nucleic acid analysis device in which circular nucleic acid measurement regions are disposed.

FIG. 7 is a flow chart showing general procedures for real-time base extension reaction.

FIG. 8 shows an example in which an unnecessary base extension reaction occurs due to the leakage from light irradiation (for cleavage of a protecting group attached for suppressing the initiation of the reaction) to fields other than the visual field.

FIG. 9 is a schematic view showing an example of a nucleic acid analysis device.

FIG. 10 is an example in which a real-time base extension reaction is controlled by delivering a solution only to a predetermined visual field through the control of the amount of the solution to be introduced.

### DESCRIPTION OF SYMBOLS

101 . . . nucleic acid analysis device
102 . . . metal structure
103 . . . total reflection prism
104 . . . excitation light laser
105 . . . excitation light irradiation region
106 . . . detector
107 . . . imaging lens
108 . . . fluorescence wavelength filter
109 . . . nucleic acid measurement region
201 . . . temperature control unit
202 . . . reagent storage unit
203 . . . dispensing unit
204 . . . solution delivering tube
205 . . . waste solution tube
206 . . . waste solution container
207 . . . two-dimensional sensor camera
208 . . . analysis computer
209 . . . apparatus control computer
210 . . . excitation light laser unit 1
211 . . . excitation light laser unit 2
212 . . . λ/4 wave plate
213 . . . mirror
214 . . . dichroic mirror.
215 . . . measurement optical path
216 . . . objective lens
217 . . . filter
218 . . . imaging lens
219 . . . camera controller
220 . . . analysis apparatus
301 . . . new excitation light irradiation region
401 . . . nucleic acid analysis device with reagent flow channel
402 . . . inlet port
403 . . . reagent flow channel 1
404 . . . nucleic acid measurement region
405 . . . outlet port
406 . . . excitation light
407 . . . reagent flow channel 2
408 . . . reagent flow channel 3
409 . . . reagent flow channel 4
601 . . . nucleic acid measurement region
602 . . . excitation light irradiation region
603 . . . reagent flow channel
604 . . . inlet port
711 . . . immobilization step for immobilizing template DNA to nucleic acid analysis device
712 . . . set step for setting nucleic acid analysis device to apparatus
713 . . . supply step for supplying reaction reagent
714 . . . shift step for shifting to next visual observation field
715 . . . observation step for observing reaction initiation and base extension reaction
716 . . . determination step for determining if the observation of all visual fields is completed
717 . . . washing step for washing nucleic acid analysis device
718 . . . removal step for removing nucleic acid analysis device
801 . . . nucleic acid analysis device
802 . . . flow channel
803 . . . inlet
804 . . . outlet
805 . . . reaction spot group
806 . . . irradiation field
807 . . . visual observation field
901 . . . reaction spot group
902 . . . visual observation field
903 . . . irradiation field
1001 . . . reagent solution drainage area
1002 . . . visual observation field
1003 . . . irradiation field
1004 . . . reaction spot group

### MODE FOR CARRYING OUT THE INVENTION

The nucleic acid analysis device according to an embodiment is a reaction device having a plurality of nucleic acid measurement regions, wherein one nucleic acid measurement region is disposed so that it is at a sufficient distance from the other nucleic acid measurement regions so that the other nucleic acid measurement regions do not enter an irradiation region. In other words, it can be said that the nucleic acid analysis device is characterized by having a plurality of nucleic acid measurement regions, and blank portions that have no reaction spot between the nucleic acid measurement regions and by its illumination of one nucleic acid measurement region with a light source. Through nucleic acid analysis using the nucleic acid analysis device according to an embodiment, since unreacted nucleic acid measurement regions are not principally irradiated with excitation light, noise information against a base sequence to be determined can be reduced and fluorescence signals from individual target nucleic acids immobilized in reaction spots within a target nucleic acid measurement region can be reliably observed. Also, the nucleic acid analysis device according to an embodiment can be installed in an analysis apparatus such as a nucleic acid analysis apparatus and used for genetic testing, for example.

Here the term “nucleic acid measurement region(s)” refers to a region(s) having one or a plurality of reaction spots in which a target nucleic acid such as a target DNA molecule is immobilized and a reaction for nucleic acid analysis is performed.

The nucleic acid analysis device according to an embodiment is produced by providing nucleic acid measurement region(s) on a substrate. Examples of substrates are not particularly limited and include substrates made of material such as quartz or silicon.

Regarding a nucleic acid measurement region(s), only one nucleic acid measurement region is provided within an irradiation region to be irradiated with excitation light, and nucleic acid measurement regions are disposed on the substrate so that they are at a sufficient distance from each other so that the other nucleic acid measurement regions do not enter the irradiation region. A blank portion having no reaction spot is present between the nucleic acid measurement regions. When image acquisition is performed using a highly sensitive camera with a ½-inch two-dimensional image sensor of a currently available CCD or CMOS imaging device and an about ×40 objective lens, about a 140 μm square (that is, about a 20000-μm² region) can be observed. This suggests that when the size of a single visual field is an about a 140 μm square, pixels of an imaging device will never go to waste. The size of a nucleic acid measurement region in the nucleic acid analysis device according to an embodiment is preferably substantially the same as that of the visual measurement field of such an optical detection system. Therefore, the size (long side or greatest dimension) of a nucleic acid measurement region on a substrate ranges from 50 μm square to 10 mm square, and it is particularly preferably 140 μm square, for example. Also, examples of the shapes of nucleic acid measurement regions include squares, quadrangles, and circles.

Meanwhile, a visual measurement field of the above size (that is, an about a 140 μm square) is irradiated uniformly with a laser beam. Moreover, a nucleic acid measurement region is disposed so as to avoid the effects of excitation light in neighboring visual fields. For these purposes, the intervals between adjacent nucleic acid measurement regions are determined in view of laser beam irradiation distribution, ranging from 1 μm to 10 mm and preferably ranging from 50 μm to 200 μm, for example. In particular, the intervals between nucleic acid measurement regions are desirably determined to have a value corresponding to single visual field (that is, about 140 μm). Such a width is determined in view of the intensity uniformity within a laser irradiation region to be used herein and desired excitation light intensity. For example, when a single nucleic acid measurement region is illuminated with a laser as light for illumination, the laser diameter and the dimension of a blank portion are limited to dimensions that allow neighboring nucleic acid measurement regions to avoid being irradiated with light that leaks. Alternatively, a nucleic acid measurement region group consisting of a predetermined number of nucleic acid measurement regions can also be irradiated with a light source.

Meanwhile, all reaction spots of a nucleic acid measurement region to be observed may be contained within a light irradiation region to which illumination intensity required for nucleic acid analysis is applied with light for illumination such as a laser, for example. Alternatively, all light irradiation regions to which illumination intensity required for measurement is applied may be contained within a nucleic acid measurement region to be observed.

When a laser is used as light for illumination, only a predetermined visual observation field (visual measurement field) can be irradiated with a laser homogenizer as explained in the following Embodiment 4.

Nucleic acid measurement regions are disposed in every direction in a grid on a substrate, such that about 10 nucleic acid measurement regions are disposed vertically and about 10 nucleic acid measurement regions are disposed horizontally. In addition, the number of nucleic acid measurement regions on a substrate is preferably determined in view of the throughput for observation of a reaction and the number of exchange of nucleic acid analysis devices according to an embodiment per nucleic acid analysis, so that the properties and usability of a nucleic acid analysis apparatus can be improved to the highest degree. Also, nucleic acid measurement regions may be disposed on a reagent flow channel, as explained in the following Embodiment 2.

In nucleic acid measurement regions, reaction spots are present. The number of reaction spots in one nucleic acid measurement region ranges from 100 to 10⁸ and preferably ranges from 10⁴ to 10⁶.

In a nucleic acid measurement region, a target nucleic acid is immobilized on reaction spots. Examples of a target nucleic acid include DNA, RNA, and PNA (peptide nucleic acid). Examples of a method for immobilization of a target nucleic acid onto a reaction spot include methods using antigen-antibody binding, binding of a tag with a substance binding thereto such as a His-Tag (histidine tag)/nitrilotriacetic acid (NTA) or iminodiacetic acid (IDA) and GST-Tag (glutathione S-transferase tag)/glutathione, avidin-biotin binding, or the like. For example, a target nucleic acid is specifically immobilized on reaction spots using biotin-avidin binding (biotin is bound to either a reaction spot or a target nucleic acid, and avidin is bound to the other). Also, an adsorption-preventing molecule is immobilized at regions other than the nucleic acid measurement region on a substrate so as to prevent unnecessary adhesion of the target nucleic acid. An example of such an adsorption-preventing molecule is, but is not particularly limited to, PLL-g-PEG described in Non-patent document 2. For example, according to the method described in Non-patent document 2, an electrode of a desired pattern is provided on a substrate, PLL-g-PEG is applied to the entire substrate surface, voltage is applied to the electrode so as to remove PLL-g-PEG on the electrode part, and then a target nucleic acid is immobilized within the region from which PLL-g-PEG has been removed. Alternatively, according to the method of using the lithography technique using near-field scanning light described in Non-patent document 3, for example, a target nucleic acid can be immobilized on reaction spots.

Furthermore, when a metal structure is used as described in Patent document 1, the metal structure is formed only within nucleic acid measurement region(s). Specifically, for example, in the case of gold, a target nucleic acid can be immobilized on a metal structure via gold-thiol binding. As described above, through immobilization of a target nucleic acid on a metal structure, fluorescence from a fluorescent molecule incorporated into the target nucleic acid to be detected upon nucleic acid analysis can be enhanced. Also, when such a metal structure is made of a rare metal, reaction spots in the nucleic acid analysis device according to an embodiment can be efficiently used. Thus, the consumption of such a rare metal can be further reduced compared with a conventional nucleic acid analysis device.

Also, for example, a nucleic acid analysis device can have a nucleic acid probe having a photodegradable substance that inhibits a nucleic acid extension reaction and a reaction field region (nucleic acid measurement region) in which a plurality of the nucleic acid probes are disposed. As explained in the following Embodiment 4, a photodegradable substance (protecting group that can be cleaved by light irradiation) is bound to a nucleic acid probe, the substance is cleaved by UV irradiation, and thus a base extension reaction is initiated. With the use of this method, a base extension reaction is inhibited at a stage at which no UV irradiation is performed, and the reaction can be initiated by UV irradiation. Examples of a photodegradable substance include caged compounds such as a 2-nitrobenzyl type, a decyl phenacyl type, or a coumarynylmethyl type (Patent document 2). The term “caged compound” is a generic name for a bioactive molecule that has been modified with a photodegradable protecting group so as to tentatively lose its activity. The generic term, “caged compound(s)” refers to a molecule(s) with bioactivity that is caged and caused to sleep.

To conduct nucleic acid analysis, a nucleic acid analysis apparatus is provided with a nucleic acid analysis device produced as described above. The apparatus can comprise, in addition to a nucleic acid analysis device, a means for supplying fluorescence-labeled primers, dNTP (N denotes A, C, G, or T), and the like to a nucleic acid analysis device, a means for irradiating the nucleic acid analysis device with light, a light emission detection means for measuring fluorescence of fluorescent molecules (with which a primer or dNTP is labeled) resulting from hybridization to a target nucleic acid or a nucleic acid extension reaction on the nucleic acid analysis device. Furthermore, the apparatus has reaction solution flow channels and a solution delivering mechanism capable of delivering a solution to a predetermined nucleic acid measurement region of the nucleic acid analysis device.

Base sequence information on a target nucleic acid can be obtained by the nucleic acid analysis apparatus according to an embodiment. For example, a solution containing a primer labeled with a fluorescent molecule is supplied to a nucleic acid measurement region on a nucleic acid analysis device. Subsequently, the fluorescent molecule is incorporated into the target nucleic acid as a result of hybridization of the target nucleic acid with the primer. The nucleic acid measurement region is irradiated with excitation light suitable for the fluorescent molecule with which the primer is labeled, fluorescence is detected, and thus the hybridization can be confirmed. Furthermore, a solution containing dNTP that is labeled with a fluorescent molecule having fluorescence properties (fluorescence wavelength or excitation wavelength) differing from those of the fluorescent molecule with which polymerase (e.g., DNA polymerase, RNA-dependent DNA polymerase (reverse transcriptase), RNA polymerase, and RNA-dependent RNA polymerase) and a primer are labeled is supplied to a nucleic acid measurement region. Thus, a base extension reaction takes place. Subsequently, the nucleic acid measurement region is irradiated with excitation light suitable for the fluorescent molecule with which dNTP is labeled, and then fluorescence is detected. Based on the fluorescence, the base information of the target nucleic acid can be obtained.

Also, with the use of the nucleic acid analysis device according to an embodiment, all base extension reactions on reaction spots can be completely observed. The nucleic acid analysis device according to an embodiment can be applied to single molecule DNA sequencing wherein a rare or the only one DNA fragment is a target nucleic acid. Furthermore, with the use of the nucleic acid analysis device according to an embodiment, a base extension reaction of the target nucleic acid is performed with a real-time scheme, and thus base sequence information can also be obtained.

Hereinafter, preferred embodiments for implementing the present invention are described with reference to the attached drawings. Here, each embodiment is an example of typical embodiments of products or methods relating to the present invention, and is not intended to limit the scope of the present invention.

### Embodiment 1

In this embodiment, an example of a nucleic acid analysis device and an example of an optical detection system in a single molecule nucleic acid analysis apparatus to which plasmon resonance is applied are explained as follows.

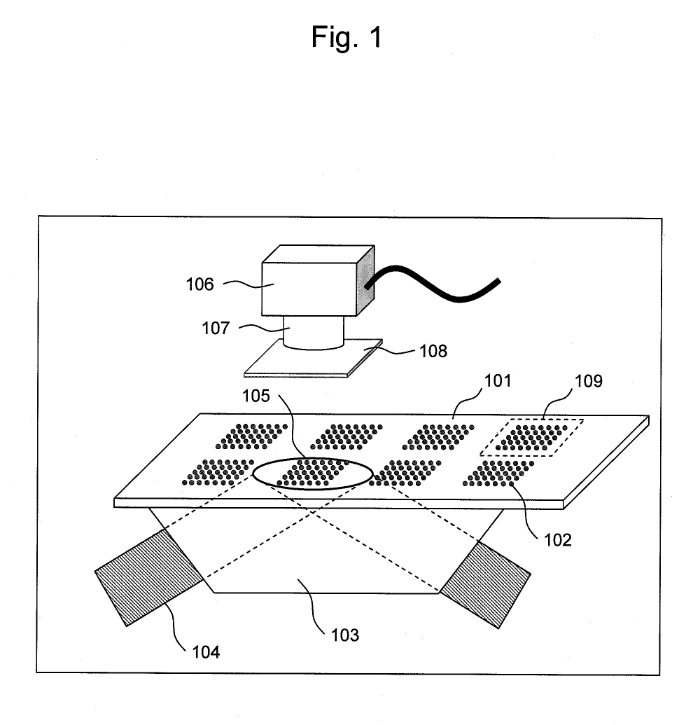

FIG. 1 shows an example of the embodiment. A nucleic acid analysis device 101 is produced using material such as quartz or silicon as a substrate. On the substrate made of the material, a metal structure 102 is divided and generated into a plurality of nucleic acid measurement regions. For the structure, material such as gold, silver, aluminum, or an alloy is used. Also, the shape of the structure may be varied, such as having the shape of beads or the shape of kernals of corn. The height of the metal structure ranges from about several tens to several hundreds of nm, for example. Also, a target DNA molecule (a target nucleic acid) to be used for a base extension reaction is immobilized on the metal structure via protein binding or another method.

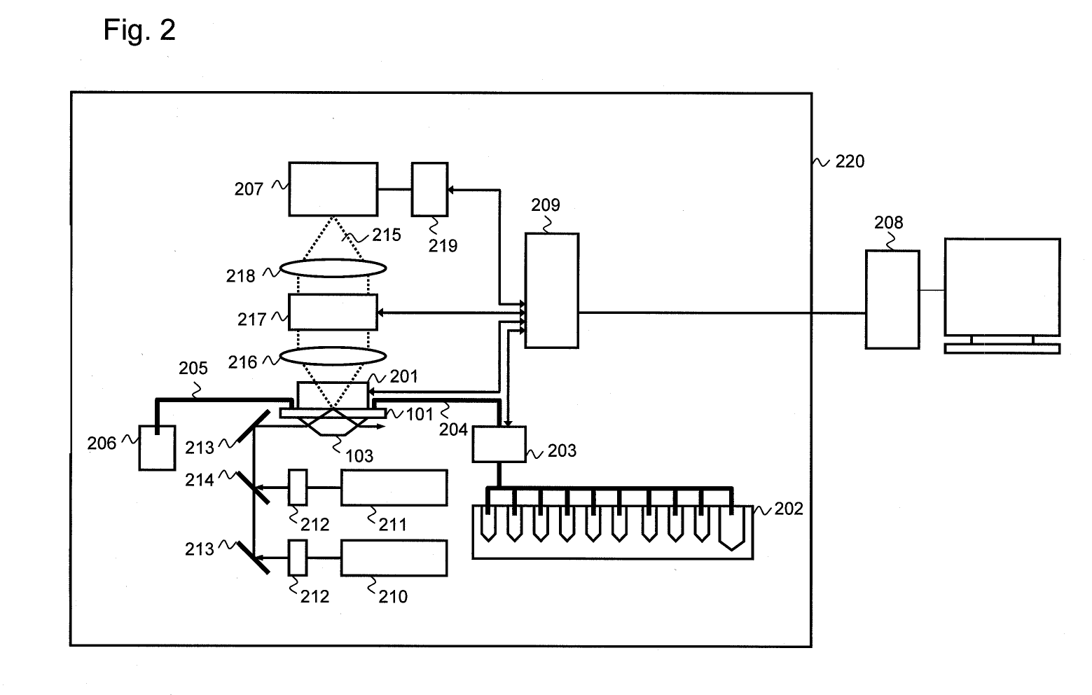

Also, FIG. 2 shows an example of an apparatus provided with a nucleic acid analysis device for determination of a base sequence. The apparatus shown in FIG. 2 is an example of a single molecule DNA sequencer, consisting of an analysis apparatus 220 and an analysis computer 208. In the analysis apparatus 220, a reaction in the nucleic acid analysis device 101 is observed with a two-dimensional sensor camera 207. A reagent is supplied to the nucleic acid analysis device 101 as follows. A reagent stored in each container within a reagent storage unit 202 is dispensed by a dispensing unit 203 and supplied by a solution delivering tube 204. The temperature of the supplied reagent is appropriately regulated by a temperature control unit 201, so that it is an optimum temperature for performing the reaction. A waste solution is discarded after completion of the reaction to a waste solution container 206 via a waste solution tube 205.

In the apparatus shown in FIG. 2, when measurement is performed with evanescent light, such as when evanescent light is optically bound to a total reflection prism 103, a nucleic acid analysis device is subjected to illumination by total reflection illumination using an excitation light laser 104. The excitation light laser 104 illuminates only one nucleic acid measurement region in an instance of measurement. In an excitation light irradiation region 105, total reflection takes place on a refractive index boundary plane on the upper substrate surface side, during which electromagnetic waves penetrate the interior on the low medium side only at a height of about 1 wavelength of incident light. Accordingly, an extremely limited region alone including the metal structure 102 is illuminated. The region is referred to as an “evanescent field.”

Also, when a base extension reaction is allowed to proceed on the nucleic acid analysis device, fluorescence incorporated into the target DNA molecule immobilized on the metal structure 102 can be measured. The fluorescence is captured as a two-dimensional image by an optical detection system consisting of a fluorescence wavelength filter 108 (that is, an optical filter that allows the transmission of only fluorescence wavelength), an imaging lens 107, and a detector 106.

The present embodiment is most significantly characterized in that the metal structures 102 are separately disposed within each nucleic acid measurement region 109. The nucleic acid measurement regions 109 are disposed at intervals so as not to affect the other nucleic acid measurement regions when a specific nucleic acid measurement region is illuminated within the excitation light irradiation region 105. In the embodiment, nucleic acid measurement regions are disposed at intervals of 300 μm in the laser irradiation direction and at intervals of 100 μm in the direction perpendicular to the laser irradiation. In addition, the distance is determined such that the irradiation intensity distribution of a laser to be used herein is sufficient for excitation of a fluorescent dye to be observed and the intervals are maintained so as not to affect neighboring visual measurement fields.

In the apparatus shown in FIG. 2 having the constitution described above, when a primer labeled with a fluorescent molecule is introduced onto the nucleic acid analysis device 101 via solution exchange to a specific concentration, the single fluorescence-labeled primer molecule hybridizes to only the target DNA molecule that is immobilized on the metal structures 102 and is complementary thereto. At this time, the fluorescent molecule is present in the evanescent field and thus is excited by evanescent light to emit fluorescence. The fluorescence is enhanced by the metal structures 102 and is captured as a two-dimensional image by the detector 106 through the fluorescence wavelength filter 108 and the imaging lens 107.

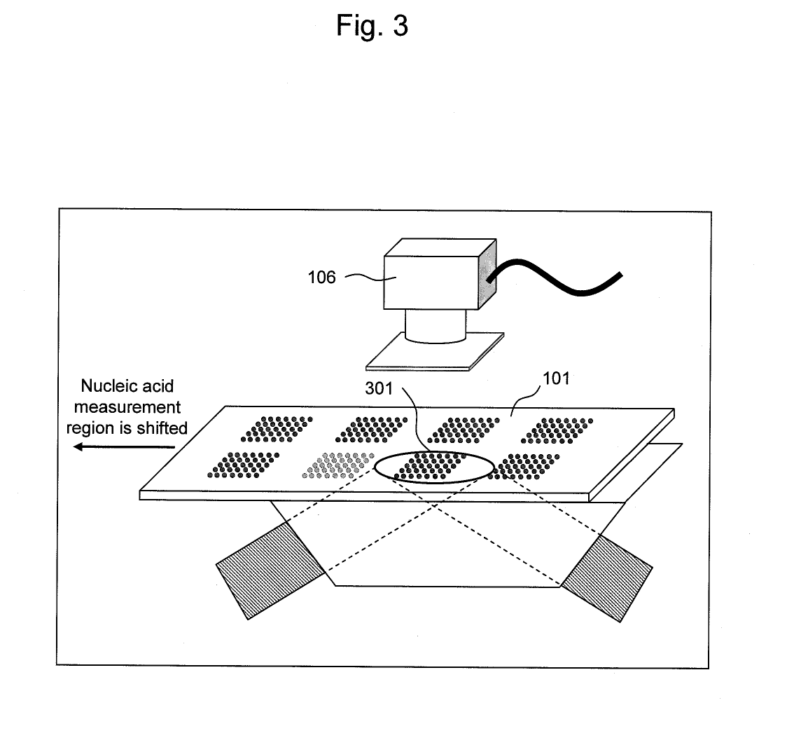

Next, FIG. 3 shows how the other nucleic acid measurement regions are measured in the nucleic acid analysis device. FIG. 3 shows a nucleic acid analysis device 101 that is shifted to cause the detector 106 to capture the next nucleic acid measurement region when it is compared with FIG. 1. The nucleic acid analysis device 101 is desirably shifted while maintaining the device with an X-Y electric stage or the like, and it is desirably designed to be automatically controllable. Through a shift of the nucleic acid analysis device, the visual field is shifted to a new excitation light irradiation region 301, and thus a nucleic acid measurement region to be measured can be shifted without removing the device.

As explained above, with the use of a scheme for subsequently measuring each nucleic acid measurement region using a nucleic acid analysis device, irradiation with light for illumination is possible only upon measurement while excluding metal structures within immeasurable regions.

Moreover, through repetition of shifting and measurement, all nucleic acid measurement regions on the nucleic acid analysis device 101 are measured. At the stage at which all measurements are completed, measurement of single base extension is completed. Thereafter, the dNTP type within a primer is changed subsequently in order of A, C, G, and then T. Solutions containing such primers are each subjected to a nucleic acid analysis device. Every time such a solution is subjected to the device, measurement and shifting of all nucleic acid measurement regions are repeated, so as to cause a base extension reaction to proceed and to determine the base sequence of a target DNA molecule.

### Embodiment 2

In this embodiment, measurement of a plurality of types of sample is explained.

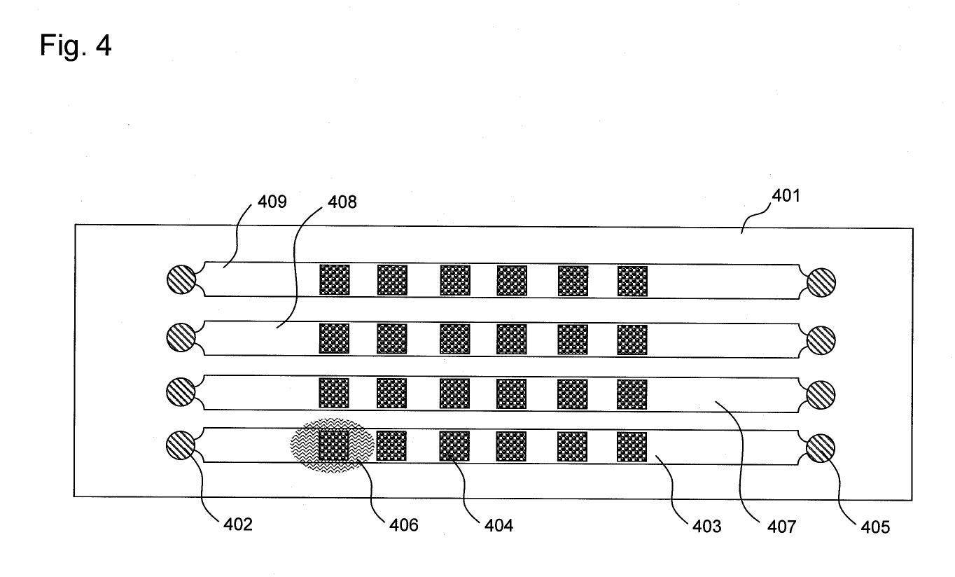

FIG. 4 is a schematic view illustrating an example of the structure of a nucleic acid analysis device, having reagent flow channels therein.

A nucleic acid analysis device 401 with reagent flow channels shown in FIG. 4 has reagent flow channels 403 having inlet ports 402 and outlet ports 405 on both ends. Also, nucleic acid measurement regions 404 are disposed between both ends of the reagent flow channel.

The nucleic acid measurement regions 404 within the reagent flow channels 403 are subjected to treatment of surfaces to which a target DNA molecule is adsorbed, according to the method for accelerating specific adsorption as described in Non-patent document 2 or 3 above (specifically, a method using PLL-g-PEG). Alternatively, regions other than the nucleic acid measurement regions 404 may be treated by techniques for chemical, photochemical, or electromagnetic non-specific adsorption prevention treatment or physical substrate surface modification treatment, so as to prevent the adsorption of the target DNA molecule.

In the present embodiment, a reagent containing a target DNA molecule having a linker for specific adsorption is injected from the inlet ports 402 into the nucleic acid analysis device 401. The target DNA molecule is specifically adsorbed to only the nucleic acid measurement regions 404 as a result of the surface treatment. After a sufficient amount of the target DNA molecule is immobilized, a washing liquid is injected from the inlet ports 402 and then the reagent is discharged. Furthermore, a primer labeled with a fluorescent molecule is introduced from the inlet ports 402 to a certain concentration via solution exchange, the single fluorescence-labeled primer molecule hybridizes to only the target DNA molecule complementary to the primer molecule. After hybridization is performed sufficiently, a washing liquid is injected from the inlet ports 402 and then the primer is discharged.

Next, each nucleic acid measurement region is irradiated with excitation light 406 and then fluorescence is measured. After completion of the measurement, excitation light is irradiated to a degree such that fluorescence is sufficiently photobleached, and thus quenching of fluorescence is caused to take place within the measurement region. At the stage at which measurement of all measurement regions is completed, measurement for single base extension is completed. Thereafter, the dNTP type within a primer is changed subsequently in order of A, C, G, and then T. Solutions containing such primers are each subjected to a nucleic acid analysis device. Every time such a solution is subjected to the device, measurement and shift of all nucleic acid measurement regions are repeated, so as to cause a base extension reaction to proceed and to determine the base sequence of a target DNA molecule.

The present embodiment wherein the nucleic acid analysis device has reagent flow channels makes it possible to analyze a plurality of different samples (that differ by reagent flow channels) using reagent flow channels (e.g., reagent flow channel 403/reagent flow channel 407/reagent flow channel 408/reagent flow channel 409) as shown in FIG. 4 without exchanging the whole device, for example. Alternatively, reaction and observation are performed using arbitrary reagent flow channels from among these reagent flow channels, the use thereof is temporarily stopped, and then measurement can be restarted using unused reagent flow channels. In this case, the device desirably has a mechanism such that irreversible marking is performed so as to be able to discriminate used reagent flow channels from unused reagent flow channels, so that unused regions can be distinguished from used regions upon restart.

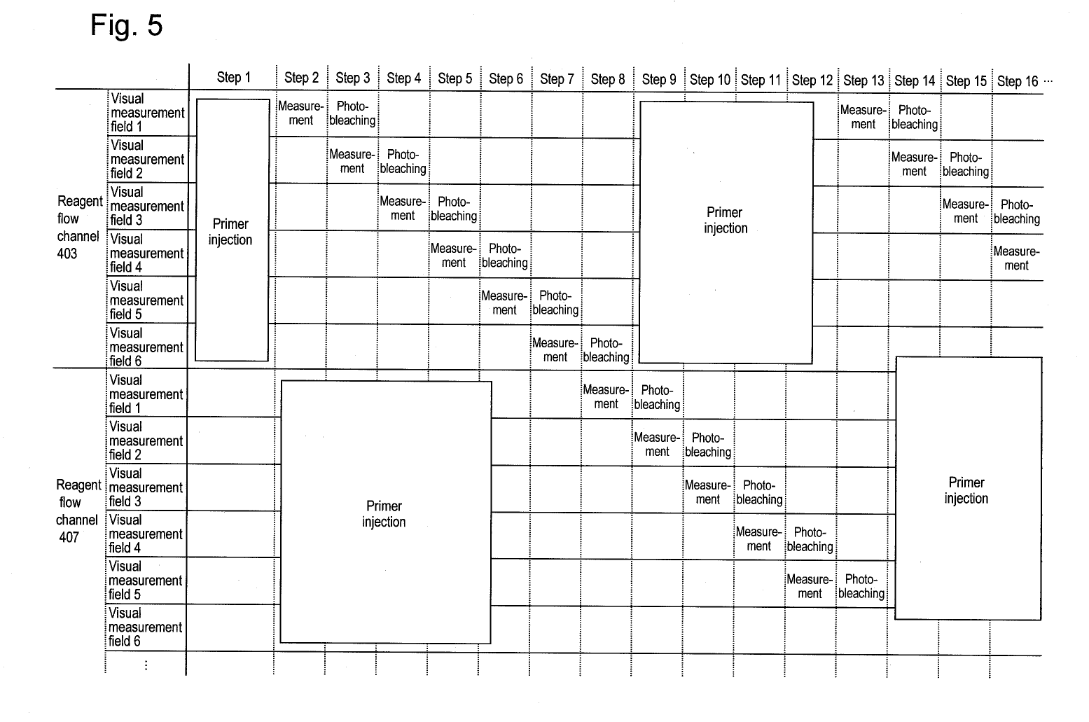

Furthermore, the present embodiment is characterized in that when a reaction that requires preparation and/or aftertreatment is performed, such treatments can be performed using unobserved reagent flow channels. FIG. 5 shows as the present embodiments, examples of parallel treatment steps using a plurality of reagent flow channels.

FIG. 5 shows examples in which upon repeated treatment with base extension reaction, reagent flow channels 403 and 407 shown in FIG. 4 are used, and six visual measurement fields (nucleic acid measurement regions 404) contained in each reagent flow channel are measured in sequence.

First, in step 1, primer injection into the reagent flow channel 403 is performed. Neither measurement nor photobleaching can be performed during primer injection. After completion of primer injection, measurement in visual measurement field 1 is initiated as step 2. On the other hand, measurement or photobleaching is not performed in the reagent flow channel 407, primer injection can be performed independently from the reagent flow channel 403.

Furthermore, in step 3, while visual measurement field 2 is subjected to measurement in the reagent flow channel 403, photobleaching is performed in visual measurement field 1, during which primer injection can be continued in the reagent flow channel 407. As described above, steps 2-7 are performed for the reagent flow channel 403, and primer injection can be performed in the reagent flow channel 407 in parallel with these steps. After completion of the measurement of visual measurement field 6 in the reagent flow channel 403, as step 8, photobleaching is subsequently performed in visual measurement field 6 of the reagent flow channel 403, simultaneously with measurement in visual measurement field 1 of the reagent flow channel 407. Therefore, all visual measurement fields corresponding to a single base extension in the reagent flow channel 403 are completed.

On and after step 9, injection of a primer containing the next dNTP type is initiated, during which measurement and photobleaching can be performed in the reagent flow channel 407 as step 9 to step 12.

With the above steps, the time required for primer injection can be shortened and base sequences can be determined with even higher throughput.

### Embodiment 3

In the present embodiment, another example of the disposition of nucleic acid measurement regions in the nucleic acid analysis device is as described below.

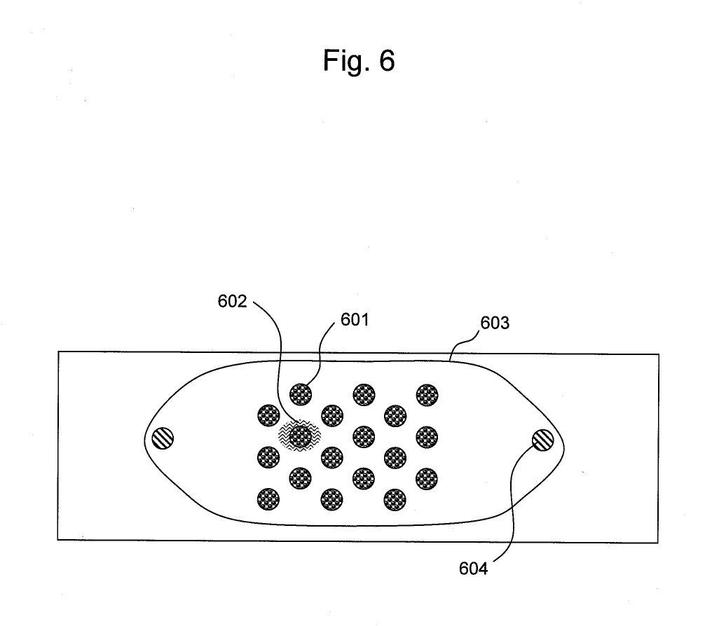

FIG. 6 shows an example of the nucleic acid analysis device in which circular nucleic acid measurement regions are disposed.

In an optical system for measuring a reaction in square or rectangular nucleic acid measurement regions, the resolution of the peripheral part may be insufficient depending on the performance of the optical system. In such a case, circular nucleic acid measurement regions are employed, observation is made for sites other than the peripheral site where the performance is decreased, and thus observation results with even higher quality may be obtained.

In the present embodiment, as shown in FIG. 6, circular nucleic acid measurement regions 601 are provided and rows of nucleic acid measurement regions are disposed alternately. Such disposition can improve the density upon disposition of the visual field.

According to the nucleic acid analysis device shown in FIG. 6, an excitation light irradiation region 602 irradiated with a laser beam does not overlap with anteroposterior nucleic acid measurement regions. Also, nucleic acid measurement regions can be disposed with even higher density, compared with a case in which nucleic acid measurement regions are aligned in matrix. The nucleic acid analysis device in the present embodiment has a reagent flow channel 603 and an inlet port 604 and can measure base extension reaction by a method similar to that employed in Embodiment 1.

### Embodiment 4

The present embodiment is an embodiment using a real-time extension reaction system, wherein in the single molecule nucleic acid analysis apparatus shown in Embodiment 1, dNTP molecules are continuously incorporated into the extending chain of the primer molecule. In real-time DNA sequencing analysis described in Non-patent document 4, four types of nucleotide having different fluorescent dyes are supplied, so as to cause successive nucleic acid extension reactions to take place without washing. When a nucleotide with a fluorescent dye attached to the phosphoric acid site is used, the phosphoric acid site is cleaved after extension reaction, so that fluorescence measurement can be performed serially without quenching. The resulting fluorescence is observed serially, so that a so-called real-time reaction scheme can be realized. Also, JP Patent Application No. 2009-266920 (applied by the applicant) more specifically describes protocols for a real-time single molecular sequencing reaction. Since a base extension reaction proceeds simultaneously with the introduction of necessary reagents in these methods, solution delivery should be controlled for each visual field or the next substrate should be set after a first measurement is completed.

In such a case, an example of a method for topically controlling the initiation of base extension reaction is a method that involves disposing a protecting group cleavable by light irradiation at position 3′ of a probe, as described in Patent document 2. According to Patent document 2, a caged compound is disposed as a protecting group at position 3′ on the oligo probe side, the protecting group is cleaved by UV irradiation, and thus a real-time base extension reaction is initiated. With the use of this method, a base extension reaction is inhibited at the stage at which no UV irradiation is performed, and the reaction can be initiated by UV irradiation.

In the case of a reaction system in which base extension is initiated by light irradiation, only a visual observation field (visual measurement field) should be irradiated with light while light is prevented from leaking to the other parts. The nucleic acid analysis device according to the embodiment is effective for such purpose.

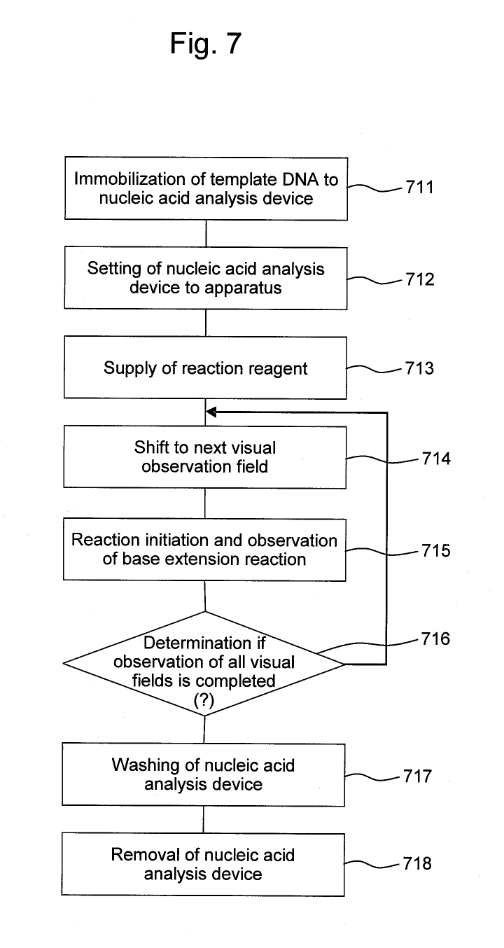

FIG. 7 shows general procedures for a real-time base extension reaction. Specifically, FIG. 7 shows procedures when the above protecting group cleavable by light irradiation is disposed for the real-time base extension reaction described in Non-patent document 4. Each step in FIG. 7 is as described below.

In an immobilization step 711 for immobilizing a template DNA onto a nucleic acid analysis device, a template DNA, primers, and an enzyme are immobilized onto the nucleic acid analysis device. Biotin-avidin binding, thiol-gold chemical binding, or the like can be used for such an immobilization method. Also, as previously described in Background Art, a technique that involves regularly disposing beads, metal structures, or the like in advance on a substrate, and immobilizing a template DNA thereon has already been commercialized.

In set step 712 for setting the nucleic acid analysis device to an apparatus, the nucleic acid analysis device treated as described above is set to an apparatus with which fluorescence can be observed using evanescent light as excitation light as described in Embodiment 1. At this time the connection of a solution delivering system, focus adjustment for the observation optical system, and the like are completed in advance.

A supply step 713 for supplying a reaction agent is a step for delivering a reaction reagent to flow channels of a nucleic acid analysis device. During the step, fluorescence labeled dNTP is applied to initiate a base extension reaction. The dNTP to be used herein has a structure wherein a phospholink nucleotide is linked to the terminal phosphoric acid, so that an enzyme cleaves the fluorescent dye in the process of base incorporation. When the protecting group attached for the inhibition of the initiation of the reaction is cleaved by light irradiation, base extension reactions are serially performed and thus every time when a base is incorporated, fluorescence labeling the nucleotide is detected.

Next, a shift step 714 for shifting (from a current observation field) to the next visual observation field (visual measurement field) is a procedure for sequentially shifting visual observation fields on a nucleic acid analysis device having a plurality of visual observation fields. Examples of such a method for shifting visual fields include a method that involves shifting a nucleic acid analysis device using an XY stage and a method that involves moving an observation optical system. In association with the shifting of visual fields, readjustment of the focus of an optical system may be required.

Subsequently, an observation step 715 for observing reaction initiation and base extension reaction is performed. When light is irradiated for cleaving a protecting group, a real-time base extension reaction is initiated. The fluorescence signals of the real-time base extension reaction are consecutively observed and then the base sequence information is collected. A visual field should be fixed until the completion of single real-time base-extension sequencing. The time required for single sequencing is thought to range from about 0 to 60 minutes based on the time taken for the loss of the enzyme activity.

When the enzyme activity is lost to make the observation of the extension reaction difficult, a determination step 716 for determining if the observation of all visual fields is completed (?) is performed. Until the completion of the observation of all visual fields, the shift step 714 (step for shifting to the next visual observation field) to the determination step 716 for determining if the observation of all visual fields is completed (?) are repeated, so that real-time base extension reaction and observation are repeated.

After completion of the observation of all visual fields, a washing step 717 for washing the nucleic acid analysis device is performed, and then reagents and the like remaining within the nucleic acid analysis device are discharged. After completion of the treatment, a removal step 718 for removing the nucleic acid analysis device is performed.

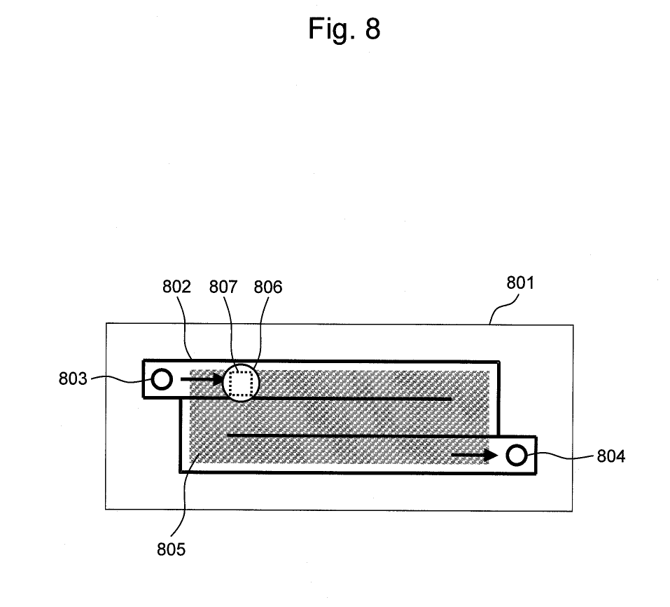

As shown in FIG. 8, the procedures outlined in FIG. 7 are performed using a nucleic acid analysis device having a reaction spot group 805 (comprising a series of reaction spots). When light irradiated for cleaving a protecting group attached for inhibition of reaction initiation leaks to other visual fields, an unnecessary base extension reaction is induced. FIG. 8 shows such a situation and specifically an example thereof wherein a flow channel 802 shown with a bold solid line is disposed in a nucleic acid analysis device 801 in which the reaction spot group 805 is disposed. A reagent (solution) is delivered from an inlet 803, the reagent flows in the direction of arrow, and then the reagent is ejected from an outlet 804. During the observation step 715 for observing reaction initiation and base extension reaction shown in FIG. 7, a visual observation field 807 shown with a broken line is observed within a circular irradiation field 806 as shown in FIG. 8, for example. A real-time base extension reaction is performed for irradiation field portions protruding from the visual observation field 807, and these are regarded as out-of-observation regions. Thereafter, when shifting from a current region to the adjacent region is achieved by the shift step 714 for shifting to the next visual observation field via the determination step 716 for determining if the observation of all visual fields is completed ?, no real-time base extension reaction is observed in a reaction spot in which a reaction has already taken place because of the irradiation field portions protruding from the relevant visual observation field.

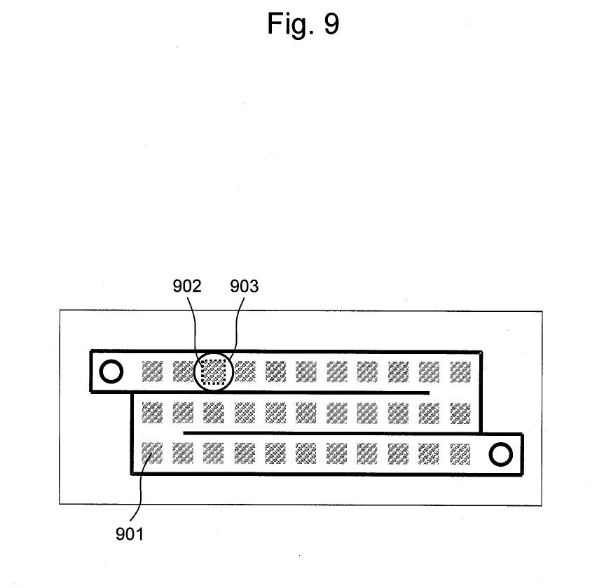

FIG. 9 shows the nucleic acid analysis device according to an embodiment. A reaction spot group 901 is divided into reaction spots having the same size as or being slightly wider than that of a visual observation field 902 shown with a broken line. Also, an irradiation field 903 indicated as a circle has a size that enables irradiation of at least entire reaction spot group 901 to be observed. The intervals between reaction spot groups are specified to be spaced such that when the irradiation field 903 encompassing a reaction spot group 901 is irradiated, no other reaction spot groups are irradiated at the same time. As a result, the irradiation field 903 partially protrudes from the region of the reaction spot group 901. However, since the reaction spot group 901 is divided for each visual field, the other reaction spot groups remain unaffected.

In addition, when a laser is used as irradiation light, a laser beam-irradiation field can be rectangular-shaped or square-shaped by a technique of laser homogenization. In this case, the intervals of reaction spot groups 901 can be narrowed as long as the irradiation field does not affect the neighboring visual fields.

### Embodiment 5

Regarding the real-time base extension reaction, Embodiment 4 describes an example of controlling reaction initiation through disposition of a protecting group cleavable by light irradiation. Meanwhile, in a reaction system not using any protecting group, a reaction is initiated simultaneously with the introduction of a solution containing primers labeled with fluorescent molecules. In the case of such a reaction system, a real-time base extension reaction proceeds even on reaction spots that have not yet been observed. Hence, the reaction system cannot be used in flow channels described in Embodiment 4. Reaction initiation should be controlled by devising a solution delivery method in the case of the reaction system wherein the reaction proceeds simultaneously with solution introduction. The nucleic acid analysis device according to the embodiment is also effective for such a case.

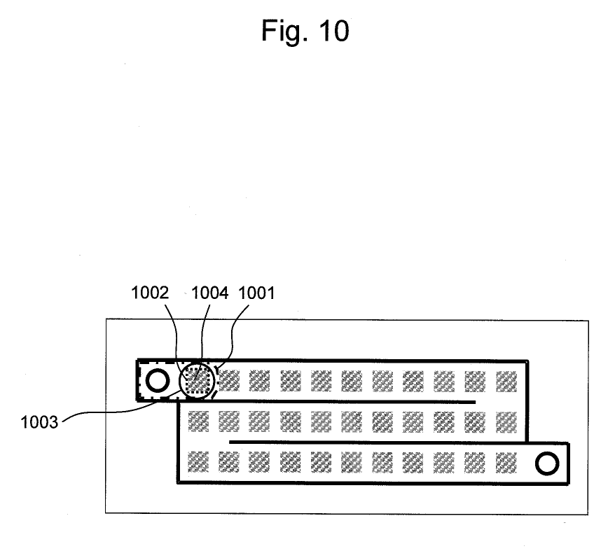

FIG. 10 shows an example of an improved version of Embodiment 4, wherein the amount of a solution to be introduced is regulated so that the solution is delivered only to a predetermined visual field, and thus the real-time base extension reaction is controlled. At the initial state, the nucleic acid analysis device is dry or is filled with a buffer solution. The amount of the introduced reagent solution is regulated, and the solution is delivered to a predetermined reaction spot group 1004. When the surface of the nucleic acid analysis device is dry, the reagent solution in a reagent solution drainage area 1001 flows in concave or convex form within the flow channel, depending on the wetting property and the like within the flow channel. When the nucleic acid analysis device is filled with a buffer solution, a slight air layer is disposed so as to prevent a reagent solution from mixing with the buffer solution, and then the reagent solution is introduced. FIG. 10 is an example in which the solution flows (or moves forward) in convex form within the flow channel. When the reagent solution flows (or moves forward) within the flow channel and reaches the reaction spot group 1004, a real-time reaction is immediately initiated. Because of this, it is desirable that the irradiation field 1003 be irradiated in advance with fluorescence excitation light, that observation of the regions within the visual observation field 1002 be initiated, that a reagent solution be delivered, and the liquid end of the reaction solution be caused to reach the reaction spot group 1004. When reaction control is performed by regulating the amount of a solution to be introduced for the nucleic acid analysis device containing a series of reaction spot groups as shown in FIG. 8, real-time extension reactions proceed in reaction spots other than the visual observation field 807, and thus the dNTP of the introduced reagent are consumed. On the other hand, in the case of the nucleic acid analysis device as shown in FIG. 10, no real-time extension reactions take place in regions other than the reaction spot group 1004, since the reagent solution does not reach such regions. Hence, reaction spot groups are disposed at sufficient intervals in view of variations in wetting due to the reagent solution, so that unintentional real-time extension reaction can be inhibited. In this case, the intervals between reaction spot groups are specified to be at a distance from each other such that when the reaction spot group 1004 within the irradiation field 1003 is irradiated, the other reaction spot groups are not irradiated, and so that the reagent solution does not come into contact with the neighboring reaction spot groups depending on variations in wetting of the reagent solution.

All publications, patents, and patent applications cited herein are incorporated herein by reference in their entirety.

1. A nucleic acid analysis device having a plurality of nucleic acid measurement regions, wherein one nucleic acid measurement region is disposed at a sufficient distance from the other nucleic acid measurement regions so as to prevent the other nucleic acid measurement regions from entering an irradiation region.

2. A nucleic acid analysis device having a plurality of nucleic acid measurement regions and blank portions having no reaction spot between nucleic acid measurement regions, whereby one nucleic acid measurement region is illuminated with a light source.

3. The nucleic acid analysis device according to claim 1 or 2, wherein the size of the nucleic acid measurement region is substantially the same as that of a visual measurement field of an optical detection system.

4. The nucleic acid analysis device according to claim 1 or 2, wherein a laser is used as light for illumination and the laser diameter and the size of the blank portion are specified so that neighboring nucleic acid measurement regions are not irradiated with light that leaks when a single nucleic acid measurement region is illuminated.

5. The nucleic acid analysis device according to claim 1 or 2, wherein a light irradiation region, to which illumination intensity required for nucleic acid analysis is applied, contains all reaction spots of a nucleic acid measurement region to be observed.

6. The nucleic acid analysis device according to claim 1 or 2, wherein the nucleic acid measurement region to be observed contains the entirety of the light irradiation region to which illumination intensity required for measurement is applied.

7. The nucleic acid analysis device according to claim 1 or 2, wherein a laser is used as light for illumination and the range to be irradiated with light having intensity required for nucleic acid analysis contains all reaction spots of a nucleic acid measurement region to be observed.

8. The nucleic acid analysis device according to claim 1 or 2, wherein the long side or the greatest dimension of the nucleic acid measurement region ranges from 50 μm square to 10 mm square.

9. The nucleic acid analysis device according to claim 1 or 2, wherein the intervals between adjacent nucleic acid measurement regions ranges from 1 μm to 10 mm.

10. The nucleic acid analysis device according to claim 1 or 2, which has a nucleic acid probe having a photodegradable substance that inhibits a nucleic acid extension reaction and a reaction field region wherein a plurality of the nucleic acid probes are disposed.

11. The nucleic acid analysis device according to claim 1 or 2, wherein a predetermined visual observation field alone is illuminated by a laser homogenizer.

12. The nucleic acid analysis device according to claim 1 or 2, wherein the nucleic acid measurement region is disposed on a reagent flow channel.

13. A nucleic acid analysis device having a plurality of nucleic acid measurement regions and blank portions having no reaction spot between nucleic acid measurement regions, wherein a nucleic acid measurement region group consisting of a predetermined number of nucleic acid measurement regions is irradiated with a light source.

14. A nucleic acid analysis apparatus, comprising the nucleic acid analysis device according to claim 1 or 2.

15. The nucleic acid analysis apparatus according to claim 14, which has a reaction solution flow channel and a solution delivering mechanism capable of delivering a solution to a predetermined nucleic acid measurement region of the nucleic acid analysis device.

16. A nucleic acid analysis method, comprising a step of subjecting the nucleic acid analysis device according to claim 1 or 2 to nucleic acid analysis, wherein a target nucleic acid is immobilized in the nucleic acid measurement region.

17. The nucleic acid analysis method according to claim 16, wherein nucleic acid analyses in each of the nucleic acid measurement regions are conducted in parallel.
